(19) United States
(12) Patent Application Publication
(10) Pub. No.: US 2010/0247376 A1
(43) Pub. Date: Sep. 30, 2010

(54) Antibacterial toothbrush spray

(76) Inventor: Trevor Douglas Yuros, Kelowna (CA)

Correspondence Address: Antony C. Edwards, P.O. Box 26020, Westbank, BC, V4T 2G3, CA

(21) Appl. No.: 12/662,072

(22) Filed: Mar. 30, 2010

Related U.S. Application Data
(60) Provisional application No. 61/202,739, filed on Mar. 31, 2009.

(51) Int. Cl. A61L 2/18; A01N 31/00; A01P 1/00

(52) U.S. Cl. 422/28; 514/724

(57) ABSTRACT

A liquid composition for spraying upon the bristles of a toothbrush so as to deposit the composition as a spray of droplets upon the bristles of the toothbrush includes a bactericidal component which is orally safe for a user of the toothbrush to ingest, and may advantageously include purified water, tea tree oil, and alcohol.

### CROSS REFERENCE TO RELATED APPLICATION

This application claims priority from U.S. Provisional Patent Application No. 61/202,739 filed Mar. 31, 2009 entitled Antibacterial Toothbrush Spray.

### FIELD OF THE INVENTION

This invention concerns dental hygiene, and in particular a spray for sanitizing toothbrush bristles.

### BACKGROUND OF THE INVENTION

Canadian patent No. 2,204,502 which issued Jan. 29, 2008, to Hecker et al. and entitled Care Station for Toothbrushes describes a dental care station where the head part of a toothbrush and the head of a spray can are at the bottom of the station housing. The housing is a two-part case-like holding device. The spray can sits in a receptacle cavity of one part of the holding device, and the toothbrush is in the other part of the holding device, which is designed as a lateral valve. Guide rails position the toothbrush when the device is closed. The spray head of the valve of the spray can sits in a brush chamber. A spray mist is released by means of a release button which is provided on the head of the housing and can act on the bottom of the spray can. By depressing the release button, the spray mist can be directed at the bristles of the toothbrush in the brush chamber in a controlled manner. Such a device is not particularly portable and is unsuitable for travel purposes because of its size.

Canadian patent No. 2,257,457 which issued Aug. 23, 2005, to Ingemann and entitled Portable Dental Hygiene Device discloses a portable dental hygiene device which has a simple design and is secured against accidental release of the spray, especially when travelling, but still requires a carrying case for aligning a toothbrush with the spray.

U.S. Pat. No. 5,328,692 discloses a dentifrice composition having as the three main constituents an enzymatic complex, fluoroamines and demthicone. A variety of optional constituents may also be present in the dentifrice. The dentifrice composition is applied to the teeth as a spray, not to the toothbrush bristles, and is contained in a suitable container under pressure and provided with a spray nozzle so that it can be applied to the teeth and the interstices between the teeth in the form of a jet of fine droplets.

### SUMMARY OF THE INVENTION

In summary, the liquid composition for spraying upon the bristles of a toothbrush according to the present invention so as to deposit the composition as a spray of droplets upon the bristles of the toothbrush includes a bactericidal component which is orally safe for a user of the toothbrush to ingest, and may advantageously include purified water, tea tree oil, and alcohol.

In use, a toothbrush bristles are rinsed with water after each use, and then the bristles are sprayed with the composition until the bristles are saturated. Excess composition shaken from the bristles. In one embodiment the composition is spread onto the bristles as a high pressure jet so as to mechanically clean the bristles and interstices between the bristles as well as force the composition into the roots of the bristles.

The composition may be contained in a spray bottle container. The composition is sprayed from the container onto the bristles as a spray of droplets so as to deposit the composition upon the bristles of the toothbrush so that the composition may soak into and wick to the roots of the bristles.

### BRIEF DESCRIPTION OF THE DRAWINGS

FIG. 1 is in side elevation view, a toothbrush and sprayer spraying the toothbrush bristles with the spray according to the present invention.

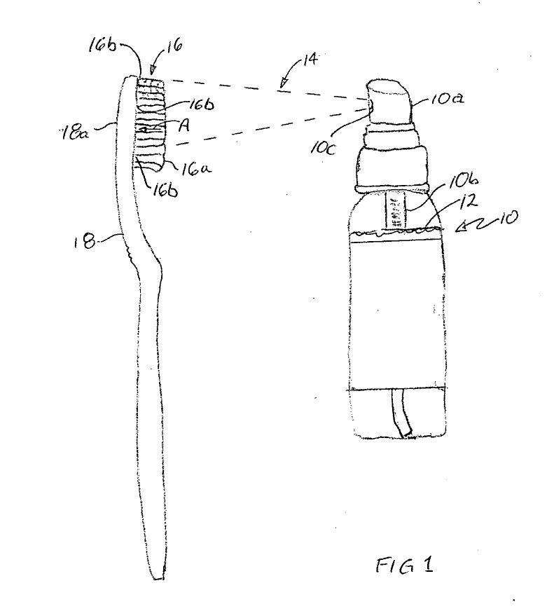

FIG. 2 is a table of ingredients of one embodiment of the composition according to a preferred embodiment.

### DESCRIPTION OF PREFERRED EMBODIMENTS

Various dentifrice compositions in the form of a paste, a gel, a powder or a solution are known which are applied with a brush to the teeth. Dentifrice tubes provided with a measuring device also have been proposed, and some of which are filled with a propellant gas to force the dentifrice from the tubes but even in this latter case a toothbrush must always be used. The use of a brush is always a constraint for many reasons, but the concern addressed in the present invention is the lack of hygiene. Specifically, when at home or travelling, typically a toothbrush is used twice a day, and once used merely allowed to air dry. The fact remains, however, that whether or not superficially dry along the exposed length of the toothbrush bristles, the bristles will harbour bacteria and the bacteria will grow in the warm wet environment provided by the bristles where they are mounted onto the head of the toothbrush. The bacteria culture is thus present when the head of the toothbrush is repeatedly replaced into the mouth of a user.

This invention eliminates this unsanitary drawback, providing a liquid dentifrice composition as described below contained in a container such as spray bottle 10 from which it is possible to eject the composition, stored as liquid 12, as a mist or as fine droplets in spray 14 (shown in dotted outline), or as a pressure jet. Spray 14 is a sanitizing liquid dentifrice solution, which is ingestible orally by a user without harming a user, and is active against bacterial cultures and which may in alternative embodiments also provide a preventive agent against dental plaque, dental decay and tartar formation, due to its composition.

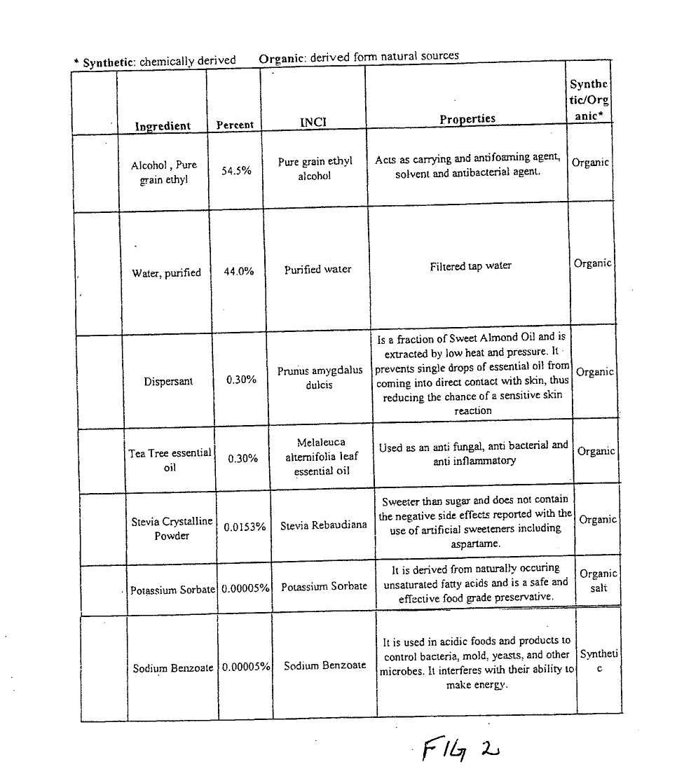

The composition of the present invention is applied as a jet or spray 14 onto the bristles 16 of a toothbrush 18 by proper orientation of for example a pump-action sprayer head 10a on container 10. The spray of the composition is sprayed onto all of the bristles 16 of the toothbrush and in particular onto the exposed ends 16a of the bristles. The sanitizing components of the composition are orally safe. For example, in one embodiment the ingredients of the composition are purified water, tea tree oil (the essential oil of Melaleuca alternifolia), stevia, and benzoic acid, sodium benzoate, polysorbate and alcohol. In a further embodiment, the ingredients of the composition are purified water, medicinal alcohol, tea tree oil, natural dispersant, stevia, potassium sorbate, sodium benzoate as set out in FIG. 2.

As illustrated, the jet or mist of the composition spray may be projected onto the bristles by a small and thus highly portable, hand-held vaporizer comprising a system 10b for pumping the liquid and expelling it outwardly through spraying nozzle 10c. The spray may also be obtained by using a canister containing composition and propellant gas under pressure, such as, for example, nitrogen gas.

In use, after each use of the toothbrush 18, the toothbrush and in particular the bristles 16 are rinsed with water and then sprayed with the composition until the bristles are at least saturated on their exposed surfaces with the composition. The user then shakes out excess liquid from the bristles, that is, shakes out the excess solution of the composition and water, and stores the toothbrush for drying as per normal. Applicant has observed that, where because of the density of bristles 16 packed onto toothbrush head 18a the sprayed-on composition does not saturate all the way down the bristles to the roots 16b of the bristles, the composition deposited on the exposed portions 16a of the bristles will wick along the bristles to the roots 16b. Thus even a cursory spraying of composition so as to soak the exposed ends and sides of the bristles, by a wicking action in direction A the composition has effect on the base and roots of the bristles where bacteria growth is prevalent.

As reported by Carson et al., (2005), RIRDC Publication No. 05/130 “Antiviral Activity of Tea Tree Oil”, a report for the Rural Industries Research and Development Corporation, Australia, tea tree oil has significant anti-viral activity against recurrent herpes labialis (cold sores) on human lips. Applicant thus believes there is a synergistic benefit to a user of the present invention in the application of the composition to the user's toothbrush; namely, not only do the bristles become sanitized of bacteria, but also the harbouring of herpes virus in the bristles may be controlled and a user of the toothbrush may then also prophylactically control the occurrence of cold sores on the user's lips by transfer of tea tree oil residue to the lips. It is the nature of the composition according to the present invention that, because the composition contains oil, merely rinsing the bristles in water does not remove the oil. A residue of oil may thus be transferred from the bristles to the user's mouth and lips. The fact that the essential oil is not dissolved in water adds to its efficacy in the composition as at least some of the tea tree oil will remain in the roots of the bristles upon rinsing of the toothbrush and continue its sanitizing effect after use and cleaning of the toothbrush, and likely ever after the bristles appear to have dried at which time, as the water evaporates, the concentration of tea tree oil will increase in the roots. The addition of a significant portion of alcohol in the composition lends great anti-bacterial effect to the composition in conjunction with the tea tree oil.

According to one embodiment of the invention, a jet of fine particle spray of the liquid composition is expelled under relatively high pressure, so as to assist the wicking by a mechanical driving of the composition in direction A towards to the roots of the bristles. This will also assist in mechanically cleaning the bristles and the interstices between the bristles.

As will be apparent to those skilled in the art in the light of the foregoing disclosure, many alterations and modifications are possible in the practice of this invention without departing from the spirit or scope thereof. Accordingly, the scope of the invention is to be construed in accordance with the substance defined by the following claims.

What is claimed is:

1. A liquid composition for spraying upon the bristles of a toothbrush as a spray of droplets so as to deposit said composition upon the bristles of the toothbrush wherein said composition includes a bactericidal component which is orally safe for a user of the toothbrush to ingest.

2. The composition of claim 1 wherein said composition includes purified water, tea tree oil, and alcohol.

3. A process for sanitizing a toothbrush comprising rinsing with water the toothbrush bristles after each use, then spraying the bristles with the composition of claim 1 until the bristles are saturated, shaking out excess said composition from the bristles.

4. The process of claim 3 wherein said composition is the composition of claim 2.

5. The process of claim 3 further comprising spraying said composition onto the bristles as a high pressure jet so as to mechanically clean the bristles and interstices between the bristles.

6. The process of claim 4 further comprising spraying said composition onto the bristles as a high pressure jet of fine particles so as to mechanically clean the bristles and interstices between the bristles.

7. An antibacterial toothbrush spray system consisting of a spray bottle container containing a liquid composition for spraying from said container onto the bristles of a toothbrush as a spray of droplets so as to deposit said composition upon the bristles of the toothbrush wherein said composition includes a bactericidal component which is orally safe for a user of the toothbrush to ingest.

8. The system of claim 7 wherein said composition includes purified water, tea tree oil, and alcohol.

9. The system of claim 8 wherein said container is further adapted to spray said composition onto the bristles as a high pressure jet so as to mechanically clean the bristles and interstices between the bristles.

10. An antibacterial toothbrush sanitizing method consisting of providing only a spray bottle container containing a liquid composition for spraying from said container onto the bristles of a toothbrush as a spray of droplets, spraying said composition from said container onto exposed surfaces of the bristles of the toothbrush and leaving said composition to soak into and wick along to the roots of the bristles, wherein said composition includes a bactericidal component which is orally safe for a user of the toothbrush to ingest.

11. The method of claim 10 wherein said composition includes purified water, tea tree oil, and alcohol.

12. The method of claim 11 further comprising spraying said composition onto the bristles as a high pressure jet so as to mechanically clean the bristles and interstices between the bristles.

13. The composition of claim 2 wherein said alcohol is substantially 55 percent of said composition, said water is substantially 44 percent of said composition, and said tea tree oil is substantially 0.3 percent of said composition.

14. The composition of claim 4 wherein said alcohol is substantially 55 percent of said composition, said water is substantially 44 percent of said composition, and said tea tree oil is substantially 0.3 percent of said composition.

15. The system of claim 8 wherein said alcohol is substantially 55 percent of said composition, said water is substantially 44 percent of said composition, and said tea tree oil is substantially 0.3 percent of said composition.

16. The method of claim 11 wherein said alcohol is substantially 55 percent of said composition, said water is substantially 44 percent of said composition, and said tea tree oil is substantially 0.3 percent of said composition.
